(19) United States
(12) Patent Application Publication
(10) Pub. No.: US 2002/0098212 A1
(43) Pub. Date: Jul. 25, 2002

(54) COSMETIC COMPOSITION CONTAINING A POLYMER AQUEOUS DISPERSION AND A DISILANOL SILICONE

(76) Inventors: CHRISTINE DUPUIS, PARIS (FR); CHRISTIANE CONDAMINE, ERAGNY (FR)

Correspondence Address: FINNEGAN HENDERSON FARABOW, GARRETT & DUNNER, 1300 I STREET NW, WASHINGTON, DC, 20005, US

(21) Appl. No.: 09/485,903

(22) PCT Filed: Aug. 18, 1998
(86) PCT No.: PCT/FR98/01817

(30) Foreign Application Priority Data
Aug. 19, 1997 (FR) 97/10.482

(51) Int. Cl. A61K 7/00; A61K 9/00; A61K 7/06; A61K 7/11; A61K 6/00

(52) U.S. Cl. 424/401; 424/47; 424/70.11; 424/70.12; 424/70.16

(57) ABSTRACT

The invention concerns an aqueous or hydro-alcoholic cosmetic composition containing an aqueous dispersion of film forming polymer insoluble particles and a non-amino &agr;,&ohgr,-disilanol silicone emulsion. The invention also concerns a method for the cosmetic treatment of keratin materials using said compositions.

The invention relates to an aqueous or aqueous/alcoholic cosmetic composition comprising an aqueous dispersion of insoluble particles of film-forming polymer and a non-aminated silicone α,ω-disilanol emulsion. The invention also relates to a process for the cosmetic treatment of keratinous substances using these compositions.

For some years, very particular interest has been displayed in the preparation of essentially aqueous hair cosmetic compositions. This is because the use of alcohol, such as ethanol or isopropanol, alone or as a mixture with a small proportion of water, can exhibit certain disadvantages, in particular an increase in flammability when the composition is in the form of an aerosol lacquer.

More generally still, the aim is to reduce the use of compounds which are volatile at atmospheric pressure, known as VOC (Volatile Organic Compounds), which are present in cosmetic compositions. VOC are mainly propellants and certain solvents, such as ethanol.

Attempts have been made, in order to decrease the amount of VOC, to replace solvents such as ethanol by water. However, while the majority of water-soluble film-forming polymers can, in solution in water, result in the production of hair fixing compositions, these compositions exhibit major disadvantages. Thus, essentially aqueous compositions of these polymers do not make it possible to obtain high degrees of fixing. Provision has certainly been made to use these water-soluble polymers at high concentrations but the increase in concentration causes such an increase in the viscosity of the compositions that satisfactory spraying can only be obtained with great difficulty. Even if correct spraying is obtained, these aqueous compositions exhibit a particularly lengthy drying time in comparison with alcohol compositions and are thus of little practical interest.

Provision has also been made to use aqueous dispersions of insoluble particles of polymers instead of polymers dissolved in aqueous, alcoholic or aqueous/alcoholic compositions.

However, to date, the results obtained are still unsatisfactory. This is because, while the fixing power is sufficient and the drying time is acceptable, the cosmetic properties are still unsatisfactory. In particular, the disentangling, softness and feel properties are unsatisfactory. Furthermore, it is difficult to remove the polymer during washing of the hair with a shampoo.

Attempts have already been made to improve the cosmetic properties of cosmetic compositions comprising a polymer dispersion without decreasing the fixing power by adding a non-functionalized silicone oil or polyoxyalkylenated silicone, but the Applicant Company has found that, in contrast to what was expected, the properties, such as disentangling, softness and feel, are not improved and are even, in some cases, debased.

The Applicant Company has now discovered that a cosmetic composition comprising, in a cosmetically acceptable medium, an aqueous dispersion of insoluble polymer particles and a non-aminated silicone α,ω-disilanol emulsion made it possible to overcome the disadvantages described above.

These compositions thus exhibit a good fixing power and good cosmetic properties, such as disentangling, softness and feel. It is easy to style or blow dry the hair after application.

The compositions according to the invention make it possible to obtain good spraying, the spray is even and the sprayed drops are fine. The compositions are distributed easily over the whole of the hair. Furthermore, surprisingly, the fixing power of the compositions is not decreased by the addition of a silicone. Finally, the drying times are low.

The subject-matter of the present invention is thus a cosmetic composition comprising, in a cosmetically acceptable medium, an aqueous dispersion of insoluble particles of polymer and a non-aminated silicone α,ω-disilanol emulsion.

The compositions according to the invention exhibit, in addition to the abovementioned advantages, good resistance to moisture, good removal on shampooing and on blow drying, and a good rate of drying.

However, other characteristics, aspects or advantages of the invention will become still more fully apparent on reading the detailed description which will follow and the concrete but in no way limiting examples intended to illustrate it.

The aqueous dispersions of insoluble particles of non-ionic or ionic polymer which can be used according to the invention are generally obtained by suspension or emulsion polymerization or copolymerization of monomers according to processes well known in the state of the art (such dispersions are also known under the name of “latex”). It is also possible to obtain aqueous dispersions of polymers by dissolving the said polymer in a water-miscible organic solvent, water is then added and, finally, the organic solvent is evaporated. This type of preparation is, for example, disclosed in French Application No. 2,697,160.

The mean diameter of the insoluble particles of polymer is generally less than 500 nm and preferably less than 250 nm. The glass transition temperature of the polymer is generally between −30EC and 150EC and preferably between 10 and 90EC.

The dispersions generally comprise at least 0.5% of surfactant making possible the dispersing and the maintenance in dispersion of the insoluble polymer. According to the invention, any type of surfactant can be used but preferably a non-ionic surfactant.

The polymer of the aqueous dispersion comprises at least one monomer chosen, for example, from styrene, butadiene, ethylene, tetrafluoroethylene, propylene, vinyltoluene, vinyl propionate, vinyl alcohol, acrylonitrile, chloroprene, vinyl chloride, vinyl acetate, urethanes, isoprene, polyols, diisocyanates, triisocyanates, isobutene, vinyl ethers, vinylpyrrolidone, vinylimidazole, trimethylammonioethyl (meth)acrylate, acrylic or methacrylic, maleic, crotonic or itaconic acids, their esters or their amides, and their mixtures.

The non-ionic polymers in the aqueous dispersions which can be used according to the present invention are, for example, chosen from the following compounds

vinyl acetate homopolymers, such as the product provided under the name of Appretan EM by the company Hoechst or the product provided under the name of Rhodopas A 012 by the company Rhône-Poulenc;

copolymers of vinyl acetate and of acrylic ester, such as the product provided under the name of Rhodopas AD 310 from Rhône-Poulenc;

copolymers of vinyl acetate and of ethylene, such as the product provided under the name of Appretan TV by the company Hoechst;

copolymers of vinyl acetate and of maleic ester, for example of dibutyl maleate, such as the product provided under the name of Appretan MB Extra by the company Hoechst;

vinyl chloride homopolymers, such as the products provided under the names of Geon 460×45, Geon 460×46 and Geon 577 by the company Goodrich;

polyethylene waxes, such as the products provided under the names Aquacer 513 and Aquacer 533 by the company Byk Cera;

polyethylene/polytetrafluoroethylene waxes, such as the products provided under the names Drewax D-3750 by the company Drew Ameroid and Wax Dispersion WD-1077 by the company R. T. Newey;

copolymers of polyethylene and of maleic anhydride;

homopolymers of alkyl acrylates and homopolymers of alkyl methacrylates, such as the product provided under the name Micropearl RQ 750 by the company Matsumoto or the product provided under the name Luhydran A 848 S by the company BASF;

copolymers of acrylic esters, such as, for example, copolymers of alkyl acrylates and of alkyl methacrylates, such as the products provided by the company Rohm & Haas under the names Primal ACZ 61 k and Eudragit NE 30 D, by the company BASF under the names Acronal 601 or Luhydraw LR 8833 or 8845, or by the company Hoechst under the names Appretan N 9213 or N 9212;

copolymers of acrylonitrile and of a non-ionic monomer chosen, for example, from butadiene and alkyl (meth)acrylates; mention may be made of the products provided under the names Nipol LX 531 B by the company Nippon Zeon or those provided under the name CJ 0601 B by the company Rohm & Haas;

styrene homopolymers, such as the product Rhodopas 5051 provided by the company Rhône-Poulenc;

copolymers of styrene and of alkyl (meth)acrylate, such as the products Mowilith LDM 6911, Mowilith DM 611 and Mowilith LDM 6070 provided by the company Hoechst, the products Rhodopas SD 215 and Rhodopas DS 910 provided by the company Rhône-Poulenc or the product Uramul SC 70 provided by the company DSM;

copolymers of styrene, of alkyl methacrylate and of alkyl acrylate, such as the product Daitisol SPA provided by the company Wackherr;

copolymers of styrene and of butadiene, such as the products Rhodopas SB 153 and Rhodopas SB 012 provided by the company Rhône-Poulenc;

copolymers of styrene, of butadiene and of vinylpyridine, such as the products Goodrite SB Vinylpyridine 2528×10 and Goodrite SB Vinylpyridine 2508 provided by the company Goodrich;

copolymers of styrene and of vinylpyrrolidone, such as the products Antara 450 and Cloud 285 provided by the company ISP;

polyurethanes, such as the products provided under the names Acrysol RM 1020 or Acrysol RM 2020 by the company Rohm & Haas or the products [lacuna] Uraflex XP 401 UZ or Uraflex XP 402 UZ by the company DSM Resins;

copolymers of alkyl acrylate and of urethane, such as the product 8538-33 [lacuna] by the company National Starch;

polyamides, such as the product Estapor LO 11 provided by the company Rhône-Poulenc.

The dispersions of insoluble particles of cationic polymer comprise, for example, the following polymers:

copolymers of acrylamide and of trimethylammonioethyl (meth)acrylate;

copolymers of alkyl methacrylate, of alkyl acrylate and of trimethylammonioethyl (meth)acrylate, such as the product Eudragit RL 30 D provided by the company Rohm Pharma.

The aqueous dispersions of insoluble particles of polymer which are particularly preferred in the context of the invention are aqueous dispersions of insoluble particles of anionic polymers.

According to the invention, it is possible, for example, to use an aqueous dispersion comprising a copolymer formed of an alkyl acrylate, of alkyl methacrylate and of one or more ethylenic carboxylic acids having from 3 to 5 carbon atoms, the alkyl radicals having from 1 to 5 carbon atoms.

The alkyl acrylate is preferably chosen from methyl acrylate, ethyl acrylate, propyl acrylate and butyl acrylate. Ethyl acrylate is particularly preferred.

The alkyl acrylate concentration is preferably between 40 and 70% by weight and more particularly between 50 and 60% by weight with respect to the total weight of the copolymer.

The alkyl methacrylate is preferably chosen from methyl methacrylate, ethyl methacrylate, propyl methacrylate and butyl methacrylate. Methyl methacrylate is particularly preferred. The alkyl methacrylate concentration is preferably between 30 and 50% by weight and more particularly between 30 and 40% by weight with respect to the total eight of the copolymer.

The preferred ethylenic carboxylic acids are acrylic acid, methacrylic acid, crotonic acid, itaconic acid or their mixtures. Acrylic acid and methacrylic acid are particularly preferred. According to the invention, it is possible to employ salts of these carboxylic acids.

The concentration of ethylenic carboxylic acids or of their salts is preferably between 5 and 15% by weight and more particularly between 8 and 12% by weight with respect to the total weight of the copolymer.

In a particularly preferred embodiment of the invention, acrylic acid is used with methacrylic acid, each in a concentration of between 2 and 10% by weight, the total of these two acids not exceeding 15% by weight of the total weight of the copolymer.

The copolymer can also comprise small amounts, that is to say less than 10%, preferably less than 5% and more particularly less than 2%, of a polymerizable monomer other than those mentioned above.

According to a particularly preferred embodiment of the invention, use is made of a copolymer comprising from 50 to 60% by weight of ethyl acrylate, from 30 to 40% by weight of methyl methacrylate, from 2 to 10% by weight of acrylic acid and from 2 to 10% by weight of methacrylic acid, the total concentration of acrylic and methacrylic acid not exceeding 15% by weight with respect to the total weight of the acrylic copolymer.

Such a copolymer is, for example, disclosed in Patent Application EP-A 590,604, which is here included here by way of reference.

An aqueous dispersion of the acrylic copolymer described above comprising 25% by weight of an ethyl acrylate/methyl methacrylate/methacrylic acid/acrylic acid copolymer is sold in particular under the tradename Amerhold DR-25 by the company Amerchol.

According to the invention, it is also possible to use an aqueous dispersion of hydroxyethyl methacrylate/methyl methacrylate/methacrylic acid/butyl acrylate copolymers, such as, for example, the product sold by the company Seppic under the name Acudyne 255.

According to the invention, it is also possible to use an aqueous dispersion of ethyl acrylate/methacrylic acid/t-butyl acrylate copolymers, such as, for example, the product sold by the company BASF under the name Luvimer Low Voc.

According to the invention, it is also possible to use an aqueous dispersion of methyl methacrylate/acrylic acid/butyl acrylate copolymers, such as, for example, the product sold by the company National Starch under the name Balance 055.

The concentration by weight of the particles of insoluble polymer in the compositions according to the invention is preferably between 1 and 35% with respect to the total weight of the composition, preferably between 5 and 20% by weight.

The emulsions comprising non-aminated silicone α,ω-disilanols which can be used in the context of the present invention can be chosen from all those already known per se.

According to the invention, non-aminated silicone α,ω-disilanol denotes any silicone not comprising at least one primary, secondary or tertiary amine or one quaternary ammonium group.

The mean size of the silicone particles in the emulsion is preferably between 1 and 10 microns and more particularly between 10 nm and 1 micron.

The emulsions which can be used according to the invention can be microemulsions, that is to say thermodynamically stable emulsions.

The emulsions are generally aqueous and comprise, in addition to water and the non-aminated silicone α,ω-disilanol, one or more surfactants. These surfactants can be of any type and more particularly of non-ionic or cationic type.

Thus, according to the present invention, it is possible to use any silicone α,ω-disilanol known per se, whether it is a silicone oil, a silicone resin or even a silicone gum. Silicones are organosilicon polymers or oligomers with a branched or crosslinked, linear or cyclic structure, of variable molecular weight, obtained by polymerization and/or polycondensation of suitably functionalized silanes and essentially composed of a repetition of main units in which the silicon atoms are connected to one another via oxygen atoms (siloxane bond), optionally substituted hydrocarbon radicals being directly bonded via a carbon atom to the said silicon atoms. The commonest hydrocarbon radicals are alkyl radicals and in particular the methyl radical, fluoroalkyl radicals, aryl radicals and in particular the phenyl radical, and alkenyl radicals and in particular the vinyl radical; other types of radicals capable of being bonded, either directly or via a hydrocarbon radical, to the siloxane chain are in particular hydrogen, halogens and in particular chlorine, bromine or fluorine, thiols, alkoxy radicals, polyoxyalkylene (or polyether) radicals and in particular the polyoxyethylene and/or polyoxypropylene radical, hydroxyl or hydroxyalkyl radicals, acyloxy or acyloxyalkyl radicals, or anionic groups, such as carboxylates, thioglycolates, sulphosuccinates, thiosulphates, phosphates and sulphates, this list, of course, being in no way limiting (so-called “organomodified” silicones). The number-average molecular weight of the silicones which can be used according to the invention can vary between 100 and several million, preferably between 1000 and 1,000,000. According to the present invention, it is possible, of course, either to use one and the same silicone or to employ several different silicones.

These silicones can be crosslinked.

Mention may in particular be made, as examples of silicones which can be used in the compositions according to the invention, of polydialkylsiloxane α,ω-disilanols, polyalkylarylsiloxane α,ω-disilanols and polydiarydialkylsiloxane α,ω-disilanols.

The alkyl groups preferably have from 1 to 4 carbon atoms and the aryl groups are preferably phenyl groups.

According to a particularly preferred embodiment of the present invention, the silicones used are chosen from polydimethylsiloxane α,ω-disilanols.

Such products are, for example, the non-ionic aqueous emulsion containing polydimethylsiloxane α,ω-disilanols sold under the name Siltech E-2170 by the company Siltech.

The silicone or silicones are present in the compositions in accordance with the invention in proportions generally of between 0.05 to 10% by weight, preferably from 0.1 to 3% by weight, with respect to the total weight of the composition.

The cosmetically acceptable continuous medium acting as vehicle in the compositions according to the invention is aqueous or aqueous/alcoholic and preferably composed of water or a mixture of water and of cosmetically acceptable solvents, such as monoalcohols, polyalcohols and glycol ethers, which can be used alone or as a mixture. More preferably still, the said vehicle is essentially composed of water.

The pH of the compositions according to the invention is generally between 2 and 9 and in particular between 3 and 8. It can be adjusted to the desired value by means of basifying or acidifying agents commonly used in cosmetics for this type of application.

When the composition according to the invention is pressurized in aerosol form, the aerosol comprises the composition described above, known as a dispensable, and at least one propellant, which can be chosen from volatile hydrocarbons, such as n-butane, propane, isobutane, pentane, chlorinated and/or fluorinated hydrocarbons and their mixtures. It is also possible to use, as propellant, carbon dioxide gas, nitrous oxide, dimethyl ether, nitrogen, compressed air and their mixtures.

In such a system, the concentration of propellants is generally between 5 and 90% and preferably between 10 and 50% by weight with respect to the total weight of the pressurized composition and more particularly between 15 and 35% by weight.

According to this preferred embodiment of the invention, the concentration of polymer particles is at least 3% by weight with respect to the weight of the pressurized composition (dispensable+ propellant) and more preferably still between 5 and 35% by weight.

The compositions according to the invention (in the pressurized or non-pressurized state) can also comprise surface-active agents, preservatives, sequestering agents, softeners, fragrances, colorants, viscosity-modifying agents, foam-modifying agents, antifoaming agents, pearlescent agents, moisturizing agents, antidandruff agents, antiseborrhoeic agents, sunscreens, ceramides, proteins, vitamins, plasticizers, hydroxy acids, electrolytes, natural or synthetic oils and waxes, fatty alcohols, esters of polyhydric alcohols, mono-, di- or triglycerides, water-soluble polymers or mixtures of these various compounds.

Of course, a person skilled in the art will take care to choose the optional compound or compounds to be added to the composition according to the invention so that the advantageous properties intrinsically attached to the composition in accordance with the invention are not, or not substantially, detrimentally affected by the envisaged addition.

The compositions according to the invention are, for example, rinse-out or leave-in hair compositions and preferably leave-in hair,compositions.

They are more particularly hair setting lotions, blow-drying lotions, fixing compositions (lacquers) and styling compositions. The lotions are packaged in various forms, in particular in vaporizers, pump-action sprays or in aerosol containers, in order to provide for application of the composition in vaporized form.

A further subject-matter of the invention is a process for the cosmetic treatment of keratinous substances, such as hair, characterized in that it consists in applying, to the keratinous substances, in particular by spraying or vaporization, a cosmetic composition as defined above and in then optionally rinsing with water, optionally after leaving for a period of time.

### EXAMPLE 1

A composition A according to the invention was prepared and was compared with three compositions B, C and D not in accordance with the invention. The four compositions are packaged in pump-action sprays.

A panel of testers evaluated the disentangling, the softness and the feel of the hair after spraying 1 g of each of these: compositions onto locks of natural hair weighing 5 g.

The grading ranges from 0 (very bad) to 5 (excellent).

The results are collated in the table below (AM means active material):

| | A | B | C | D |
|---|---|---|---|---|
| | (Inven- | (compara- | (compara- | (compara- |
| In g AM | tion) | tive) | tive) | tive) |
| Amerhold DR | 10 | 10 | 10 | 10 |
| 25⁽¹⁾ | | | | |
| Siltech E-2170⁽²⁾ | 1 | — | — | — |
| Q2-5220⁽³⁾ | — | 1 | — | — |
| L7230⁽⁴⁾ | — | — | 1 | — |
| Ethyl phthalate | 1.5 | 1.5 | 1.5 | 1.5 |
| Water, q.s. for | 100 | 100 | 100 | 100 |
| Disentangling | 3 | 2 | 0 | 3 |
| Softness | 3 | 1.5 | 1 | 2 |
| Feel | 3 | 1.5 | 1 | 2 |

(1) Amerhold DR 25 from Amerchol: Ethyl acrylate/methyl methacrylate/methacrylic acid/acrylic acid copolymer as an aqueous dispersion comprising 25% by weight of the copolymer. (2) Siltech E-2170 from Siltech: Non-ionic aqueous emulsion comprising 60% by weight of polydimethylsiloxane α,ω-disilanols (dimethiconol). (3) Q2-5220 from Dow Corning: Polyoxyethylenated and polyoxypropylenated polydimethylsiloxane (dimethicone copolyol). (4) L7270 from OSI: Polyoxyethylenated and polyoxypropylenated polydimethylsiloxane (dimethicone copolyol).

The aqueous emulsion containing polydimethylsiloxane α,ω-disilanols (A) makes it possible to improve the softness and the feel of the hair, whereas the dimethicone copolyol (B and C) causes a decline in these two properties.

### EXAMPLE 2

Two compositions A and B according to the invention were prepared and were compared with two compositions C and D not in accordance with the invention. The four compositions are packaged in pump-action sprays.

A panel of testers evaluated the disentangling, the softness and the feel of the hair after spraying 1 g of each of these compositions on to locks of natural hair weighing 5 g.

The results are collated in the table below:

| | A | B | C | B |
|---|---|---|---|---|
| | (Inven- | (Inven- | (compara- | (compara- |
| In g AM | tion) | tion) | tive) | tive) |
| Acudyne 255⁽¹⁾ | 10 | 10 | 10 | 10 |
| Siltech E-2170⁽²⁾ | 1 | — | — | — |
| Q2-5220⁽³⁾ | — | — | 1 | — |
| TP511 A⁽⁴⁾ | — | 1 | — | — |
| Ethyl phthalate | 3 | 3 | 3 | 3 |
| Water, q.s. for | 100 | 100 | 100 | 100 |
| Disentangling | 4 | 4.5 | 2.5 | 4 |
| Softness | 3 | 3.5 | 2 | 2.5 |
| Feel | 3 | 3 | 2 | 2.5 |

(1) Acudyne 255 from Seppic: Hydroxyethyl methacrylate/methyl methacrylate/methacrylic acid/butyl acrylate copolymer as an aqueous dispersion comprising approximately 40% by weight of the copolymer. (2) Siltech E-2170 from Siltech: Non-ionic aqueous emulsion containing 60% by weight of polydimethylsiloxane α,ω-disilanols (dimethiconol). (3) Q2-5220 from Dow Corning: Polyoxyethylenated and polyoxypropylenated polydimethylsiloxane (dimethicone copolyol). (4) TP511 A from OSI: Anionic aqueous emulsion containing 35% by weight of crosslinked polydimethylsiloxane α,ω-disilanols (dimethiconol).

The aqueous emulsions containing polydimethylsiloxane α,ω-disilanols (A and B) make it possible to improve the disentangling, the softness and the feel of the hair, whereas the dimethicone copolyol (B) causes a decline in these three properties.

### EXAMPLE 3

A composition A according to the invention was prepared and was compared with two compositions B and C not in accordance with the invention. The three compositions were pressurized as aerosols.

A panel of testers evaluated the disentangling, the softness and the feel of the hair after spraying 2 g of each composition onto locks of natural hair weighing 5 g.

The results are collated in the table below (AM means active material):

| | A | B | C |
|---|---|---|---|
| In g AM | (Invention) | (comparative) | (comparative) |
| Amerhold DR | 15.4 | 15.4 | 15.4 |
| 25⁽¹⁾ | | | |
| Siltech E-2170⁽²⁾ | 1.5 | — | — |
| Q2-5220⁽³⁾ | — | 1.5 | — |
| Ethyl phthalate | 2.3 | 2.3 | 2.3 |
| Water, q.s. for | 100 | 100 | 100 |
| Disentangling | 2 | 1 | 2 |
| Softness | 2.5 | 1.5 | 2 |
| Feel | 2.5 | 1.5 | 2 |

(1) Amerhold DR 25 from Amerchol: Ethyl acrylate/methyl methacrylate/methacrylic acid/acrylic acid copolymer as an aqueous dispersion comprising 25% by weight of the copolymer. (2) Siltech E-2170 from Siltech: Non-ionic aqueous emulsion containing 60% by weight of polydimethylsiloxane α,ω-disilanols (dimethiconol). (3) Q2-5220 from Dow Corning: Polyoxyethylenated and polyoxypropylenated polydimethylsiloxane (dimethicone copolyol).

The pressurization scheme was the following:

| | |
|---|---|
| Dimethyl ether (propellant) | 35 g |
| Above composition (dispensable) | 65 g |

The aqueous emulsion containing polydimethylsiloxane α,ω-disilanols makes it possible to improve the softness and the feel of the hair, whereas the dimethicone copolyol causes a decline in these two properties.

1. Cosmetic composition, characterized in that it comprises, in a cosmetically acceptable medium, an aqueous dispersion of insoluble particles of polymer and a non-aminated silicone α,ω-disilanols emulsion.

2. Composition according to claim 1, characterized in that the said aqueous dispersion results from the polymerization or from the copolymerization of monomers chosen from styrene, butadiene, ethylene, propylene, vinyltoluene, vinyl propionate, vinyl alcohol, acrylonitrile, chloroprene, vinyl acetate, urethanes, isoprene, isobutene, vinyl ether, vinylpyrrolidone, vinylimidazole and acrylic or methacrylic, maleic, crotonic or itaconic acids, their esters or their amides.

3. Composition according to either one of claims 1 and 2, characterized in that the polymer of the aqueous dispersion is chosen from copolymers of alkyl acrylate, of alkyl methacrylate and of one or more ethylenic carboxylic acids or their salts having from 3 to 5 carbon atoms, the alkyl radicals having from 1 to 5 carbon atoms.

4. Composition according to any one of the preceding claims, characterized in that the polymer is chosen from ethyl acrylate/methyl methacrylate/methacrylic acid/acrylic acid copolymers.

5. Composition according to any one of claims 1 to 3, characterized in that the polymer of the aqueous dispersion is chosen from hydroxyethyl methacrylate/methyl methacrylate/methacrylic acid/butyl acrylate copolymers, ethyl acrylate/methacrylic acid/t-butyl acrylate copolymers and methyl methacrylate/acrylic acid/butyl acrylate copolymers.

6. Composition according to any one of the preceding claims, characterized in that the concentration by weight of the polymer particles is between 1 and 35% with respect to the total weight of the composition.

7. Composition according to any one of the preceding claims, characterized in that the non- aminated silicone α,ω-disilanol(s) are present in the proportion of 0.05 to 10% by weight with respect to the total weight of the composition.

8. Composition according to claim 7, characterized in that the silicone or silicones are present in the proportion of 0.1 to 3% by weight with respect to the total weight of the composition.

9. Composition according to any one of the preceding claims, characterized in that the silicone or silicones are chosen from polydialkylsiloxane α,ω-disilanols, polyalkylarylsiloxane α,ω-disilanols and polydiaryldialkylsiloxane α,ω-disilanols.

10. Composition according to claim 9, characterized in that the silicone or silicones are chosen from polydimethylsiloxane α,ω-disilanols.

11. Composition according to any one of the preceding claims, characterized in that the compositions are hairsetting lotions or blow-drying lotions.

12. Composition pressurized in an aerosol, characterized in that it comprises a composition as defined in any one of claims 1 to 10 and at least one propellant.

13. Composition according to claim 12, characterized in that the propellant is chosen from volatile hydrocarbons, such as n-butane, propane, isobutane, pentane or chlorinated and/or fluorinated hydrocarbons, carbon dioxide gas, nitrous oxide, dimethyl ether, nitrogen, compressed air and their mixtures.

14. Composition according to either one of claims 12 and 13, characterized in that the concentration by weight of the polymer particles is at least 3% by weight and preferably between 5 and 35% with respect to the total weight of the pressurized composition.

15. Composition according to any one of claims 12 to 14, characterized in that the propellant is present in concentrations of between 5 and 90% by weight with respect to the total weight of the pressurized composition.

16. Composition according to claim 15, characterized in that the propellant is present in concentrations of between 10 and 50% by weight with respect to the total weight of the pressurized composition.

17. Process for the cosmetic treatment of keratinous substances, such as hair, characterized in that it consists in applying, to the keratinous substances, a cosmetic composition as defined in any one of claims 1 to 16 and in then optionally rinsing with water, optionally after leaving for a period of time.
